(19) United States
(12) Patent Application Publication
(10) Pub. No.: US 2007/0173800 A1
(43) Pub. Date: Jul. 26, 2007

(54) System and method for heating a shape memory material spinal correction element for corrective spinal surgery

(75) Inventors: Marc M. Sanders, Mengelo (NL); Paul John Firkins, Worcs (GB); Kevin Michael Amesbury, Derby (GB)

Correspondence Address: LAHIVE & COCKFIELD, LLP, ONE POST OFFICE SQUARE, BOSTON, MA, 02109-2127, US

(73) Assignee: DePuy Spine, Inc., Raynham, MA (US)

(21) Appl. No.: 11/340,235

(22) Filed: Jan. 26, 2006

(51) Int. Cl. A61B 18/04

(52) U.S. Cl. 606/33

(57) ABSTRACT

A device and method of correcting a spinal defect in a patient employs the use of shape memory material. The shape memory material may be transitioned between a martensitic state and an austenitic state by applying heat to the shape memory material. Heat may be applied using conductive heat, convective heat, or electromagnetic radiation such as infrared radiation.

### FIELD OF THE INVENTION

The present invention relates to spinal correction devices used in corrective spinal surgery. More particularly, the present invention relates to devices and methods for heating a spinal correction element that is used in a corrective spinal surgery to transition a shape memory material in the spinal correction element from a martensitic state to an austenitic state to allow shaping of the spinal correction element.

### BACKGROUND OF THE INVENTION

Spinal corrective systems may be used in orthopedic surgery to correct a deformity or misalignment in the spinal column, as well as stabilize and/or fix a desired relationship between adjacent vertebral bodies. Spinal corrective surgery may be used to correct a variety of defects in the spine, such as scoliosis, spondylosis, spondylolisthesis, vertebral instability, spinal stenosis, degenerated, herniated, or degenerated and herniated intervertebral discs, as well as injuries, including fractures and torn ligaments and surgical manipulations, including laminectomies.

In spinal corrective surgery, a spinal corrective device, such as a spinal rod, and bone anchors, such as screws, bolts, wires and/or hooks, may be used to stabilize the spine, repair a defect and/or bring misaligned vertebrae back into alignment and secure the aligned vertebrae in the aligned position. A standard surgical procedure for correcting spinal defects in the current state of the art involves first distracting the vertebrae, pulling the vertebra back into alignment with the spinal column, and then stabilizing the spine using posterior spinal implants consisting of anchoring devices and spinal corrective elements. An interbody fusion device may also be used to give further stability and correction of the disc height. Compression across the vertebrae may be applied across the construct to set the correct balance of forces in the region.

A spinal corrective device used in such systems is generally a relatively rigid fixation rod or plate that is coupled to a bone by attaching the element to various anchoring devices. The corrective device can extend between two bone regions to effect stabilization, positioning, reduction and/or fixation of the bones. The spinal corrective device can have a predetermined contour that has been designed according to the properties of the target implantation site and, once installed, the spinal corrective element holds the bones in a desired spatial relationship, either until desired healing or spinal fusion has occurred, or for some longer period of time.

Shape memory materials have been used in spinal corrective systems to apply corrective forces to the spine by transitioning a shape memory material in a spinal corrective device between a flexible martensitic state and a rigid austenitic state to generate corrective forces that are transferred to the spine. Shape memory materials are characterized by an ability to restore the material to a pre-selected shape of the austenitic state after plastic deformation. For example, in an austenitic state, the shape memory material is stiff, rigid and has a set, pre-selected shape. In a martensitic state, the shape memory material becomes flexible and deformable and may have any of a variety of shapes. The microstructure of the material in the martensitic state is characterized by “self-accommodating twins”, having a zigzag arrangement, which allow for deformation of the material shape by de-twinning.

Generally, shape memory materials are induced to transition between the rigid austenitic state and the flexible martensitic state by changing the temperature, pressure, stress, chemistry and/or another parameter of the shape memory material. For example, a shape memory material may be cooled to make the material flexible in a martensite state, and subsequently heated to return the material to the original shape with the austenitic structure. The temperature at which a shape memory material starts transforming to austenite is known as the “austenite start temperature.” Further heating increases the temperature of the shape memory material to induce a complete transformation to the austenitic state, setting the shape of the material in the pre-selected shape of the austenitic structure. The temperature at which a shape memory material finishes transforming to austenite is known as the “austenite finish temperature.” A shape memory material can transition to the martensitic state to allow deformation and shaping of the spinal corrective device by changing the temperature of the material below a selected temperature (i.e., cooling the material). The temperature at which a shape memory material begins transformation to the martensite state is known as the “martensite start temperature”. Further cooling decreases the temperature of the shape memory material to induce a complete transformation to the martensite state. The temperature at which a shape memory material finishes transformation to the martensite state is known as the “martensite finish temperature.” Performing heating of shape memory material in corrective devices, such as spinal rods, from their cooled or martensitic state following implantation can be problematic. For example, it may be desirable to only transition certain sections from martensitic state to an austenitic stat. Also the temperature must be controlled to control the rate of transition from martensitic state to an austenitic state. Therefore, what is needed is a method for heating a corrective device made of shape memory materials that allows for the controlled heating of a specific area of the corrective device.

### SUMMARY OF THE INVENTION

The present invention provides system and method for heating a spinal correction device, such as a spinal rod, to an austenitic state to apply a corrective force. The system and method may allow for in-vivo shaping of the spinal correction device while inserted in the body and attached to bone anchors by cooling the spinal correction device after insertion.

In a first aspect, a method of heating a spinal fixation element comprising a shape memory material is provided. The method comprises providing a spinal correction device comprised at least partially of a shape memory material; and heating a portion of the spinal correction device to transition the portion of the shape memory material from a martinsitic state to an austenitic state.

In another aspect, another method of heating a spinal fixation element comprising a shape memory material is provided. The method comprises providing a spinal correction device comprised at least partially of a shape memory material; and applying electromagnetic radiation to a portion of the spinal correction device to transition the portion of the shape memory material from a martinsitic state to an austenitic state. In one embodiment the electromagnetic energy comprises infrared radiation.

In another aspect, a method of correcting a spinal deformity is provided. The method comprises the steps of placing a cooled spinal correction device in the body, wherein the cooled spinal fixation element includes a shape memory material in a martinsitic state; and heating a portion of the spinal correction device while in the body to transition the portion of the shape memory material form to an austentic state.

In another aspect, a device for applying heat to a segment of a spinal correction device is provided. The device comprises a handle for holding the device; and a heating element extending from the handle for applying heat to the segment of the spinal correction device;

In another aspect, a convective heating device for heating a spinal fixation element is provided. The device comprises a housing configured to be wrapped around the spinal fixation element and a path for heating gas to be circulated through the housing.

In another aspect, a method of heating a spinal correction device comprising a shape memory alloy is provided. The method comprises the steps of providing a spinal correction device comprising a shape memory material; and heating a first segment of the spinal correction device without affecting the temperature of other segments of the spinal correction device.

### BRIEF DESCRIPTION OF THE FIGURES

The foregoing and other objects, features and advantages of the invention will be apparent from the following description and apparent from the accompanying drawings, in which like reference characters refer to the same parts throughout the different views. The drawings illustrate principles of the invention and, although not to scale, show relative dimensions

FIG. 1 illustrates a spinal correction system according to one embodiment of the invention.

FIG. 2 illustrates the steps involved in performing a spinal corrective surgery according to an illustrative embodiment of the invention.

FIG. 3 illustrates an embodiment of a spinal corrective device including insulation between different segments of the device to facilitate segmental heating and/or cooling of the spinal corrective device.

FIG. 4 illustrates a electromagnetic radiation heating device for a spinal corrective device according to one embodiment of the invention.

FIG. 5A illustrates an embodiment of a conductive heating device for a spinal corrective device according to another embodiment of the invention.

FIG. 5B illustrates a another embodiment of a conductive heating device for a spinal corrective device according to another embodiment of the invention

FIG. 6 illustrates an embodiment of a convection heating device for a spinal corrective device according to another embodiment of the invention.

FIG. 7 illustrates a heating device comprising a canister of heating gas in another embodiment of the invention.

### DETAILED DESCRIPTION OF THE INVENTION

The present invention provides an improved surgical device and method for correcting a spinal defect in a patient. The present invention will be described below relative to certain exemplary embodiments to provide an overall understanding of the principles of the structure, function, manufacture, and use of the instruments disclosed herein. Those skilled in the art will appreciate that the present invention may be implemented in a number of different applications and embodiments and is not specifically limited in its application to the particular embodiments depicted herein.

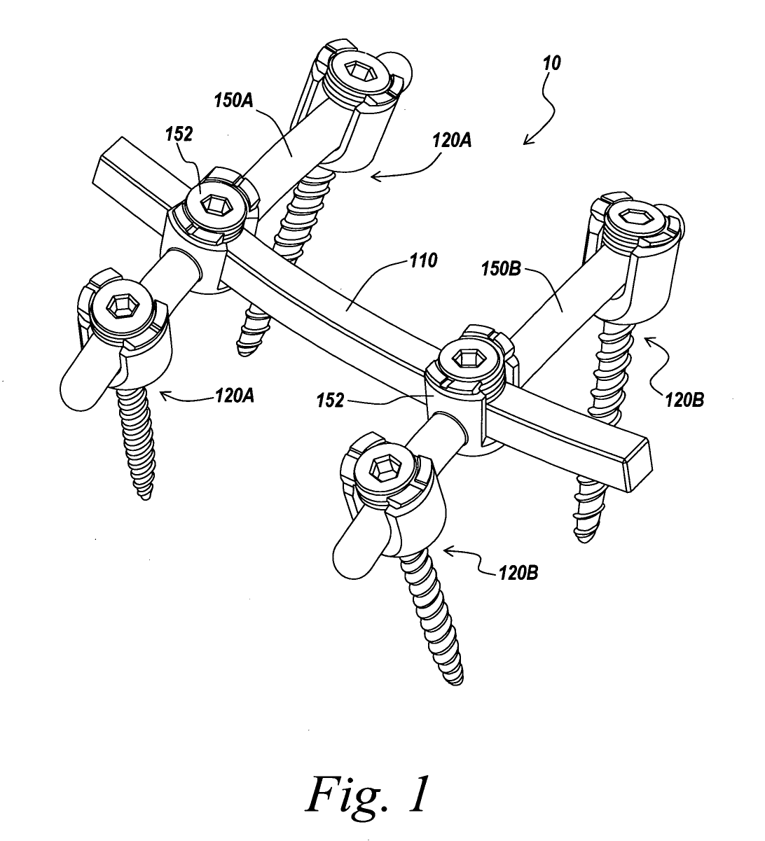

FIG. 1 illustrates a spinal correction system 10 suitable for performing a spinal corrective surgery according to an illustrative embodiment of the invention. The spinal correction system preferably includes a plurality of bone anchors, illustrated as bone screws 120, and one or more spinal corrective devices, illustrated as a spinal rod 110, for attaching to the bone anchors 120 and applying a corrective force to move the spine to a corrected shape. A first set of bone screws 120A is attached to and straddles a first vertebra, and a second set of bone screws 120B is attached to and straddles a second vertebra. Bridges 150A and 150B extend between each pair of bone screws 120A, 120B, respectively, each including a rod-receiving portion 152 for receiving the spinal rod 110 to connect the spinal rod 110 to the bone anchors and vertebrae. Locking mechanism may be provided to secure the bridges 150A, 150B to the bone anchors and the spinal corrective device to the bridges 150. Such components are known in the art and may have any suitable size, shape and configuration. Several pairs of bone anchors and bridges may be used to connect the spinal corrective device across a number of vertebrae.

The spinal corrective device 110 is formed at least partially or entirely of a shape memory material, which may be a shape memory polymer or alloy, such as nitinol (a nickel-titanium alloy), to create corrective forces that are applied to the spinal column. In the austenitic phase, the spinal rod 110 has a pre-selected shape matching a corrected or desired shape of the spine. In the martensitic phase, the spinal rod 110 is flexible and malleable to match the shape of the spine in the deformed shape. The spinal corrective device is attached to the connectors or bone anchors while in the martensitic state, and subsequently returned to the austenitic state while attached to the connectors or bone anchors to apply corrective forces to the spine and return the spine to a corrected or desired shape.

The spinal corrective element may have a circular cross-section, a polygonal-shaped cross-section or a cross-section with any other suitable shape. For the illustrative spinal rod 110 preferably has square cross-section to provide torsional correction and stability during correction of the spinal defect.

While the illustrative system comprises a single spinal rod 110, the spinal correction system 10 may alternatively comprise two spinal rods 110 or other spinal corrective elements straddling the vertebrae and connected directly to the bone anchors 120A and 120B or via intermediate connectors 152 and bridges 150.

In the illustrative embodiment of the invention the shape memory material in a spinal corrective device used for correcting a spinal defect transitions between martensitic and austenitic states by changing the temperature of the material. The spinal corrective device may be cooled to make the material flexible in a martensite state, and subsequently heated to return the material to the original shape with the austenitic structure. For example, in one embodiment, a material in the martensitic state returns to the pre-selected shape of the austenitic state by changing the temperature of the material, usually by heating the material, above an austenite start temperature, and preferably past the austenite finish temperature. The shape memory material can transition to the martensitic state to allow deformation and shaping of the spinal corrective device by changing the temperature of the material (i.e., cooling the material) below the martensite start temperature and preferably below the martensite finish temperature.

The invention provides improved methods and devices for heating a spinal corrective device, such as the spinal rods 110, to transition the material to from a martensitic state to an austenitic state returning the memory material to its original shape and applying a corrective force. The heating can be applied in-vivo, while the spinal corrective element is implanted in the patient's body. The heating can be applied along segments of the spinal corrective device, or evenly along the entire device. The system and method for heating may comprise applying a electromagnetic radiation, such as infrared radiation, to the material to induce the transition to a austenitic state.

A temperature sensor and/or a feedback mechanism may be provided to allow for control of the heating and/or cooling and/or other transition-inducing parameter. For example, a sensor may measure the temperature of the shape memory material in the spinal corrective element, which may be used to adjust the heating and/or cooling of the material to control the transition between the martensitic and austenitic states.

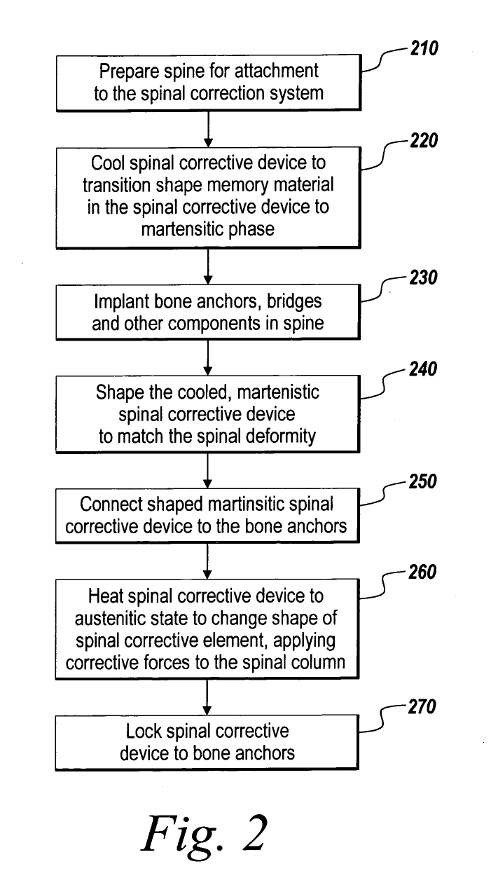

FIG. 2 illustrates the steps involved in performing a spinal corrective surgery using a spinal correction system according to an embodiment of the invention. In step 210, the spine is prepared for attachment to the spinal correction system by opening the patient to expose the spine. In step 220, one or more spinal corrective devices, such as a spinal rod 110, formed at least partially of a shape memory material, such as nitinol, are cooled to transition the spinal corrective device to a martensitic phase, making the device malleable and deformable. The device is preferably cooled below the martensite final temperature of the material, to provide sufficient time to manipulate the spinal corrective device in the martensite state, while being maintained above a certain temperature to avoid cold burns to the patient. For a nitinol material, the transition from martensitic to austenitic state and vice versa is characterized by a martensite start temperature of between about 10 and about 15 degrees Celsius, a martensite final temperature between about 10 and about 20 degrees Celsius below the martensite start temperature, an austenite start temperature of between about 0 and about 10 degrees Celsius and an austenite final temperature of about 32 degrees Celsius. For a nitinol spinal rod, the rod is preferably cooled from a temperature of about 25 degrees Celsius to a temperature of between about −20 and about −30 degrees Celsius to transition the material to the martensitic state, though one skilled in the art will recognize that the temperature to which the device is cooled may vary.

The spinal corrective device may be provided initially in the austenitic state, where the device has the shape of an expected corrected spine. The initial spinal corrective element may have any of a variety of lengths and curvatures to accommodate particular anatomical variations of the individual patient.

After cooling in step 220, the spinal corrective device 110 may be stored in ice-water or another cold environment to maintain the material in the martensite state.

As described in detail below, the spinal corrective device may be cooled in step 220 through a variety of means. For example, the spinal corrective device 110 may be cooled by applying a cold gas or other coolant directly or indirectly to the spinal corrective device. A further exploration of cooling techniques can be found in U.S. patent application DUQ-19.

In step 230, the bone anchors 120, such as polyaxial screws, and bridges 150 are implanted in the spine at selected locations for receiving the spinal corrective device 110. Alternatively, the bone anchors and bridges are implanted before the cooling of the spinal corrective device in step 220.

After the bone anchors 120 are implanted and the spinal corrective device 110 is transitioned to the martensitic state, the cooled, martensitic spinal corrective device 110 is shaped to match the spinal deformity in step 240. The spinal corrective element may be shaped manually, using benders, or using any suitable instrument. Shaping the flexible spinal corrective device to match the deformity allows the spinal corrective element to match and fit into receiving portions on the bone anchors or other components already connected to the spine.

In one embodiment, a phantom spinal rod may be used in step 240 to shape a martensitic spinal rod to match a spinal deformity. The phantom spinal rod is flexible and may be bent and/or twisted to fit into the receiving-portions to determine the shape of the deformed spine. The cooled spinal corrective device 110, which is flexible and deformable while below the martensite start temperature, and is preferably below the martensite finish temperature, is bent and/or twisted to match the phantom spinal rod, and thus the spinal deformity.

In step 250, the shaped spinal corrective device is placed in receiving portions of the bone anchors 120, and secured thereto. Approximators may be used, if needed, to approximate the spinal corrective device into the receiving portions. During step 250, the temperature of the spinal corrective device is preferably maintained at a sufficiently low temperature, below the austenite start temperature to prevent transition back to the austenite state, allowing the surgeon bend, handle and place the spinal corrective device in the patient. The shaped martensitic spinal corrective device is then fixed to the bone anchors, either directly or indirectly, to be able to withstand a selected amount of corrective force for correcting the spinal deformity.

In step 260, the spinal corrective device is transitioned to the austenitic state, which returns the spinal corrective device to the pre-selected shape of the expected corrected spine to apply corrective forces to the spinal column. In the illustrative embodiment, the spinal corrective device is heated to transition the material to the austenitic state. Preferably, the surgeon transitions the spinal corrective device to the austenitic state by heating the spinal corrective element to or past the austenitic start temperature, and preferably past the austenite finish temperature. The corrective forces in the spinal corrective device 110 are transferred through the bone anchors 120 to the spine. Due to its visco-elastic properties, the deformed spine with be corrected by the corrective forces of the system.

Any suitable means may be used to transition the spinal corrective device to the austenitic state. For example, a heater for heating a shape memory material in a spinal corrective device may employ a heated liquid that circulates near or in contact with the shape memory material to raise the temperature of the shape memory material into the austenitic state. Alternatively, a heater may employ induction heating, resistance heating, electromagnetic radiation heating and/or any other suitable means for increasing the temperature of the shape memory material. According to one embodiment, body heat may be used to partially or fully heat the shape memory material to the austenitic state. Particular exemplary means will be described in more detail below.

Preferably, the shape memory material is heated above the austenitic final temperature and above body temperature to create sufficient force to move the spine into the corrected configuration, while keeping the temperature sufficiently low (i.e., equal to or below about 40 degrees Celsius) to prevent burns to the body.

The correctional force applied to the spine to correct the spinal deformity is preferably applied evenly and spread across the vertebral bodies.

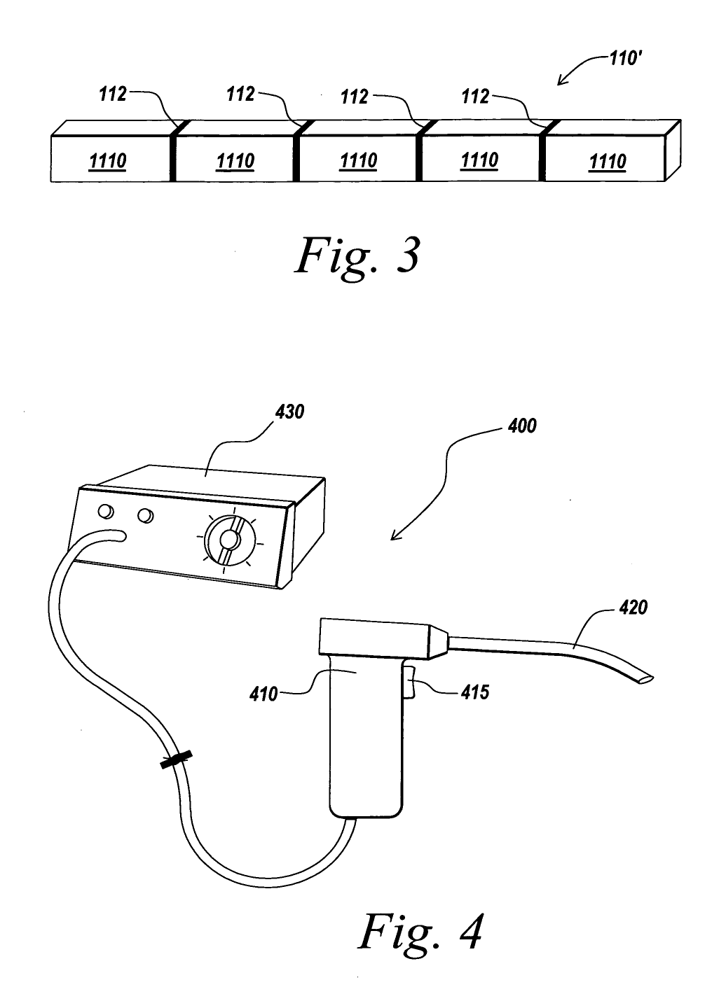

In one embodiment of the invention, isolated segments of the spinal corrective device may be controllably transitioned between the martensitic state and the austenitic state at a time. For example, as shown in FIG. 3, an embodiment of the spinal corrective device 110′ may have insulation 112 between different segments 1100 of the device to prevent heat or cooling from transmitting from one segment to another to facilitate segmental correction.

According to one embodiment, the vertebrae are fully corrected during the surgical procedure, i.e., the temperature of the shape memory material increases past the austenite final temperature during the surgical procedure, so that no additional transition occurs after surgery.

In step 270, the spinal corrective device may be locked in the anchors using set screws or another locking mechanism.

According to one aspect of the invention, the spinal corrective device 110 may be heated while disposed in the body. Providing the ability to segmentaly heat the spinal corrective device allows for control of the corrective forces applied to the spine. Heating may be controllably applied to the shape memory material to apply the corrective forces to the spine as needed.

A variety of different devices and methods may be used to heat the shape memory material of a spinal corrective device to an austenitic state in step 260. For example, in one aspect, the spinal correction device is heated by applying electromagnetic radiation, such as electromagnetic radiation to the spinal correction device.

In many instances, a heating device may be used to apply heat to the spinal correction device. An example of one such embodiment can be seen in FIG. 4. In this example the heating device is an electromagnetic radiation heating device 400 having a handle 410, a heating element 420, and a power supply 430.

In this example, the handle 410 has a pistol grip style shape. Any number of handles styles and shapes may be used. In other embodiments, the handle may provide a pen or knife style grip. Other possible shapes and configurations will be apparent to one skilled in the art given the benefit of this disclosure. The handle 410 insulates the holder from the heating element. The handle 410 of the present example also provides a trigger switch 415 for activating the heating device 400. In alternate embodiments a foot switch (not shown) may be provided for activating the heating device 400.

In this example, the heating element 420 is an electromagnetic radiation emitter, and more specifically an infrared radiation emitter. Infrared radiation is particularly useful in that it provides a focused, controlled heating mechanism. In other embodiments other types of electromagnetic radiation, such as microwave radiation, may be used. Other possible electromagnetic radiation will be apparent to one skilled in the art given the benefit of this disclosure. The electromagnetic emitter may have any number of sizes and shapes, but preferably it is sized and shaped for easy use in-vivo so as to be able to heat a segment of a spinal correction device without damaging the surrounding tissue.

The power supply 430 of the heating device 400 provides the power for generating electromagnetic radiation. The power supply 430 can be used to control the power and intensity of the electromagnetic radiation emitted by the heating device 400. This allows the heating device to be adjusted for any number of applications. Other possible features and configurations will be apparent to one skilled in the art given the benefit of this disclosure.

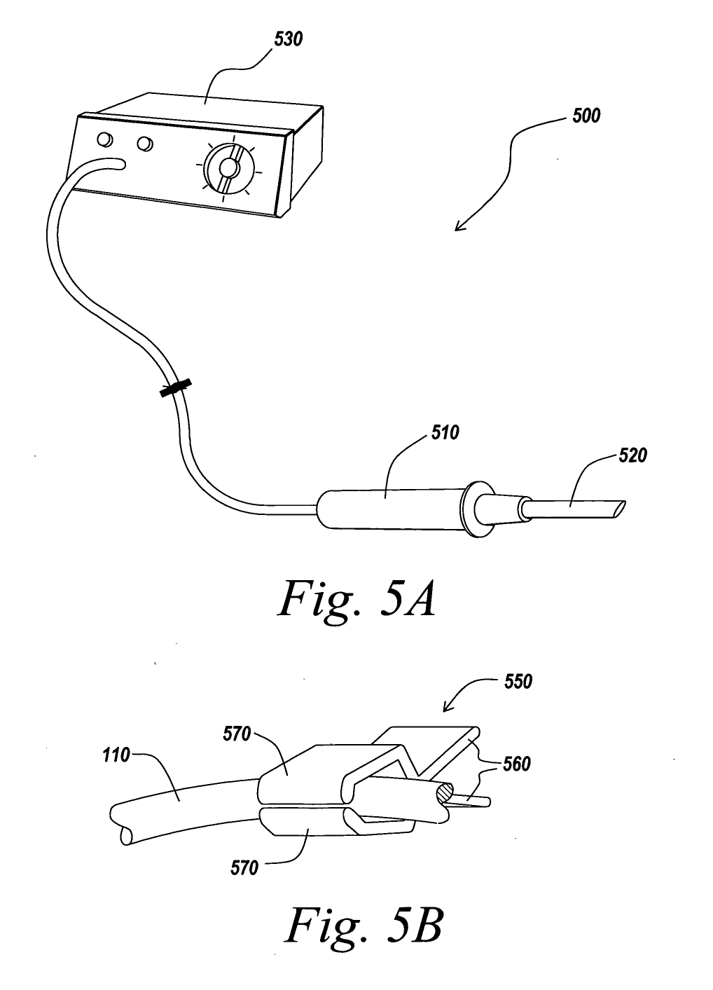

FIG. 5A is another example of a heating device 500. In this example, the heating device is a conductive heating device 500 having a handle 510, a heating element 520, and a power supply 530.

In this example, the handle 510 has a hilt style shape commonly found on cauterizing devices that allow for a pen or knife style grip. The handle 510 insulates the user holding the device from the heating element 520. Other possible shapes and configurations will be apparent to one skilled in the art given the benefit of this disclosure.

In this example, the heating element 520 is a conductive heating element, and more specifically a resistive heating element. Current is passed though the heating element causing the element to heat. In other embodiments a heated gas or fluid may be passed though the heating element causing the heating element to heat. The heating element may then be directly applied to a segment of the spinal correction device causing the segment to heat up through conduction. The conductive heating element may have any number of sizes and shapes, but preferably it is sized and shaped for easy use in-vivo so as to be able to heat a segment of a spinal correction device without damaging the surrounding tissue. In certain embodiments the heating element is configured so that it can be easily moved on or off the spinal correction device allowing for successive segments to be heated. This also allows for different segments to be heated to different extents. That is, more corrective force may be applied to some segments then others by heating the segments to a greater degree.

The power supply 530 of the heating device 500 provides the power for heating the resistive heating element. The power supply 530 can be used to control the power and intensity of the heat generated by the heating device 500. This allows the heating device to be adjusted for any number of applications. Other possible features and configurations will be apparent to one skilled in the art given the benefit of this disclosure.

FIG. 5B is another embodiment of a conductive heating device 550. In this embodiment the heating device is in a clip configuration. The heating element 570 is configured to clamp around a segment of the spinal correction device 110 while the handle 560 is used to open and close the heating element 570 for placing the heating element 570 onto the spinal correction device. Other possible configurations for conductive heating devices includes wraps and collars.

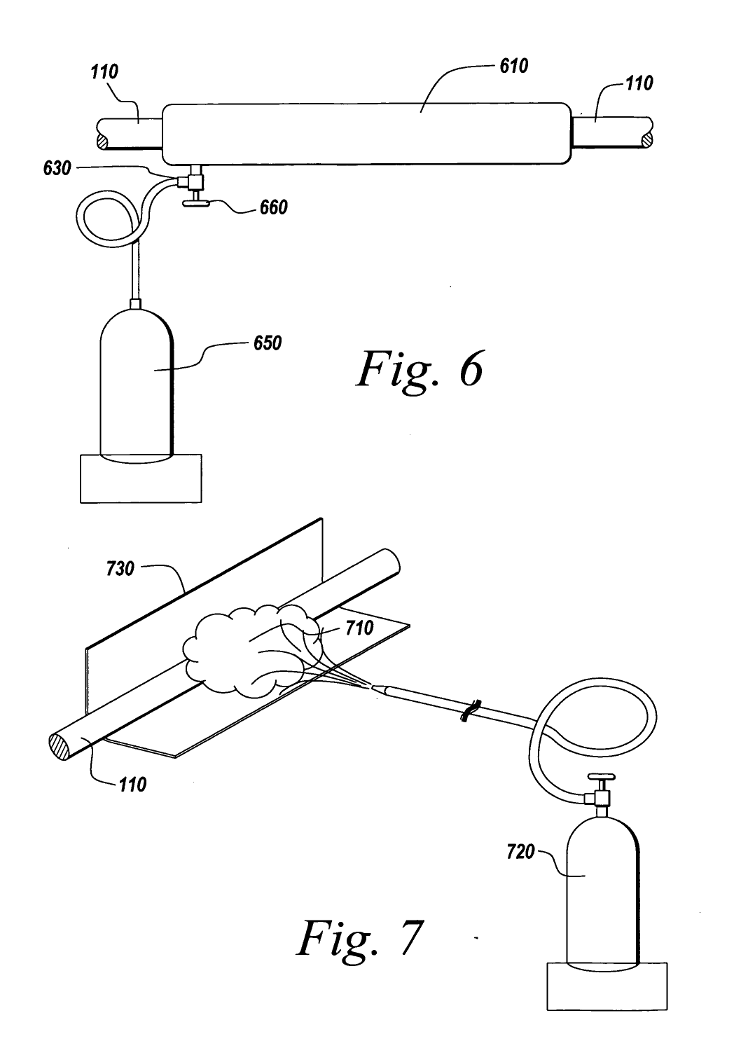

In another embodiment, a convection heating device may be used. An example of this can be seen in FIG. 6. In this example, the convection heating device 600 for heating a spinal correction device comprises a housing, jacket, or collar 610 that circulates a gas or liquid from a heating tank 650. The housing 610 may be wrapped around the location on the spinal corrective device 110 where the heating is required. A nozzle 630 on the housing is coupled to a source of liquid or gas 650. Valves 660 or other flow controllers may be used to control the flow of heating gas or liquid through the fluid paths and housing 610. When the liquid or gas is injected into the space between the housing and spinal correction device, the heat is transferred from the liquid or gas to the section of spinal correction device by convection. The housing 610 is preferably insulated. The jacket 410 may also selectively circulate a cooling fluid or gas to cool the shape memory material to a martensitic state.

The heating device may include a thermocouple or other suitable temperature sensor for monitoring the temperature of the spinal corrective device during heating. The heating process may be controlled based on feedback from the temperature sensor.

In another embodiment, a shown in FIG. 7, a heated gas 710 may be sprayed directly on a spinal corrective device 110 from a reservoir or container 720 to heat the shape memory material in the spinal corrective device. As the heated gas 710 comes into contact with spinal correction device 110 the heated gas 710 raises the temperature of the spinal correction device 110. In certain embodiments a heat shield 730 is used to protect the tissue in the area and to redirect heat back towards the spinal correction device 110. The heat shield may be made of metal, ceramic, or any other material that contains and reflects the heat from the heated gas. In some embodiments, the heat shield is configures to be insulated on the surface facing away from the spinal correction device to protect the surrounding tissue and allow for ease of handling.

The heating devices and methods depicted in FIGS. 4-7 allow for targeted heating of isolated sections of a spinal correction device. The illustrative heating devices facilitate targeted application of electromagnetic radiation, conduction heat, and convection heat to selected locations of a spinal correction device to transition only at a portion or selected portions of the spinal correction device form a martensitic state to an austenitic state.

The heating devices and method of heating provide significant advantages over prior methods for heating spinal corrective devices, including enhanced control over the heating process, the ability to provide segmental transition to the austenitic state, in-vivo heating of the spinal corrective element to enhance control over the corrective forces applied to the spine, more thorough heating of the spinal corrective element and other advantages.

The present invention has been described relative to an illustrative embodiment and application in spinal correction surgery. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

1. A method of heating a spinal fixation element comprising a shape memory material, the method comprising:
providing a spinal correction device comprised at least partially of a shape memory material; and
heating a portion of the spinal correction device to transition the portion of the shape memory material from a martinsitic state to an austenitic state.

2. The method of claim 1, wherein the heating is performed using electromagnetic energy.

3. The method of claim 2, wherein the electromagnetic energy comprises infrared radiation.

4. The method of claim 1, wherein the heating is performed using a conductive heating device.

5. The method of claim 1, wherein the heating is preformed by applying a heated gas to the spinal correction device.

6. The method of claim 1, wherein the heating is performed using a convective heating device.

7. The method of claim 1, wherein only a segment of the spinal correction device is heated.

8. The method of claim 1, wherein the entire length of the spinal correction device is heated.

9. The method of claim 1, wherein the spinal correction device has a shape of a corrected spine in the austenitic state and is adjusted to match a deformity in the spine in the martinsitic state.

10. The method of claim 9, further comprising the step of inserting a shaped spinal correction device in the martinsitic state into a patient the shaped spinal correction device in receiving portions fixed to vertebrae of a patient.

11. The method of claim 10, wherein heating the spinal correction device to transition to the austenitic state to apply corrective forces to the spine.

12. The method of claim 1, wherein the shape memory material comprises a shape memory metal.

13. The method of claim 1, wherein the shape memory material comprises nitinol.

14. A method of heating a spinal fixation element comprising a shape memory material, the method comprising:
providing a spinal correction device comprised at least partially of a shape memory material; and
applying electromagnetic radiation to a portion of the spinal correction device to transition the portion of the shape memory material from a martinsitic state to an austenitic state.

15. The method of claim 14, wherein the electromagnetic energy comprises infrared radiation.

16. A method of correcting a spinal deformity, comprising the steps of:
placing a cooled spinal correction device in the body, wherein the cooled spinal fixation element includes a shape memory material in a martinsitic state; and
heating a portion of the spinal correction device while in the body to transition the portion of the shape memory material form to an austentic state.

17. The method of claim 16, further comprising the step of adjusting the shape of the spinal correction device while in the body.

18. The method of claim 16, wherein the step of heating comprises applying electromagnetic radiation.

19. The method of claim 18, wherein the electromagnetic radiation comprises infrared radiation.

20. The method of claim 16 wherein the step of heating comprises applying a conductive heating device.

21. The method of claim 1, wherein the heating is preformed by applying a heated gas to the spinal correction device

22. The method of claim 16 wherein the step of heating comprises applying a convective heating device

23. The method of claim 22 wherein the convective heating device can be used to cool the spinal correction device.

24. A device for applying heat to a segment of a spinal correction device, comprising:
a handle for holding the device; and
a heating element extending from the handle for applying heat to the segment of the spinal correction device;

25. The device of claim 24, wherein the heating element comprises a restive element for conductively heating a segment of the spinal correction device.

26. The device of claim 24, wherein the heating element comprises an electromagnetic radiation emitter.

27. The device of claim 25, wherein the electromagnetic radiation emitter is an infrared radiation emitter.

28. A convective heating device for heating a spinal fixation element, the device comprising:
a housing configured to be wrapped around the spinal fixation element; and
a path for heating gas to be circulated through the housing.

29. A method of heating a spinal correction device comprising a shape memory alloy, the method comprising the steps of:
providing a spinal correction device comprising a shape memory material; and
heating a first segment of the spinal correction device without affecting the temperature of other segments of the spinal correction device.

30. The method of claim 29, wherein the step of heating comprises applying electromagnetic radiation.

31. The method of claim 30, wherein the electromagnetic radiation comprises infrared radiation.

32. The method of claim 29 wherein the step of heating comprises applying a conductive heating device.

33. The method of claim 29 wherein the step of heating comprises applying a convective heating device
